(19) United States
(12) Patent Application Publication
(10) Pub. No.: US 2021/0172003 A1
(43) Pub. Date: Jun. 10, 2021

(54) MOLECULAR IMAGING AND RELATED METHODS

(71) Applicant: Optical Biosystems, Inc., Santa Clara, CA (US)

(72) Inventors: Marc BEAL, Concord, CA (US); Ronald M. COOK, Novato, CA (US); Peter Jay COASSIN, Encinitas, CA (US); Arturo ORJALO, Oakland, CA (US); Jekwan RYU, Cupertino, CA (US)

(21) Appl. No.: 17/086,016

(22) Filed: Oct. 30, 2020

Related U.S. Application Data
(63) Continuation of application No. 13/999,508, filed on Mar. 4, 2014, now abandoned.
(60) Provisional application No. 61/851,276, filed on Mar. 6, 2013.

(51) Int. Cl. C12Q 1/6804; C12Q 1/6841; G01N 21/64

(52) U.S. Cl. CPC C12Q 1/6804; C12Q 1/6841; G01N 2021/6439; G01N 21/6428; G01N 21/6456

(57) ABSTRACT

A method of imaging single molecules includes exposing a test sample to a probe. The probe includes a first portion that specifically binds to a target molecule and a second portion that is detectable as the result of one or more chemical groups that interact with light at one or more wavelengths. The probe binds to a target molecule to provide a complex. The method also includes exposing the complex to one or more wavelengths of light that interact with the one or more chemical groups; and detecting a result from the interaction of the light and the one or more chemical groups to provide an image of the one or more single molecules. The image possesses a resolution better than 450 nm over a view field area of at least 1×10⁵ μm², and the image is obtained in a single detection step without variation of any detection settings.

### FIELD OF THE INVENTION

The present invention generally relates to imaging single molecules, or one or more collections of single molecules, and methods related to the imaging.

### BACKGROUND OF THE INVENTION

There have been reports of methods by which one can detect a single molecule in an extremely small area (i.e., area below 10 nm by 100 nm) at a resolution of about 300 nm. For instance, fluorescence in situ hybridization (FISH) is a method of measuring gene expression that is sensitive enough to detect single mRNA molecules. As originally described by Singer, the method involves the simultaneous hybridization of five oligonucleotide probes to each mRNA target. Femino A M, Fay F S, Fogarty K, Singer R H. Visualization of single RNA transcripts in situ. Science. 1998; 280:585-590. The oligonucleotides are each about 50-nucleotides long, and they are each labeled with up to five fluorophores. The mRNA target becomes visible as a diffraction-limited fluorescent spot upon hybridization using a fluorescence microscope.

A modified FISH method has been developed by Raj. See, Raj A, van den Bogaard P, Rifkin S A, van Oudenaarden A, Tyagi S. Imaging individual mRNA molecules using multiple singly labeled probes. Nat Methods, 2008; 5; 877-879. This method, which uses a large number of singly-labeled probes instead of a limited number of multiply-labeled probes, is used to overcome a number of issues posed by Singer's original FISH procedure: heavily-labeled oligonucleotides are difficult to synthesize and purify; when certain fluorophores are present in multiple copies on the same oligonucleotide, self-quenchings occur; signals are prone to variability. See, Femino A M, Fogarty K, Lifshitz L M, Carrington W, Singer R H. Visualization of single molecules of mRNA in situ. Methods Enzymol. 2003:361; 245-394. Also see, Randolph J B, Waggoner A S. Stability, specificity and fluorescence brightness of multiply-labeled fluorescent DN A probes. Nucleic Acids Res. 1997; 25; 2923-2929. Raj's modified method generates uniform signals that can be identified to provide accurate mRNA counts in an extremely small field of view using relatively simple probe generation and purification.

Despite the work of scientists such as Singer and Raj, there is still a need in the art for improved molecular imaging and related methods.

### SUMMARY OF THE INVENTION

In a method aspect, the present invention provides a method of imaging single molecules. The method comprises the steps of: a) exposing a test sample to a probe, wherein the probe comprises a first portion that specifically binds to a target molecule and a second portion that is detectable as the result of one or more chemical groups that interact with light at one or more wavelengths, wherein the probe binds to a target molecule to provide a complex; b) exposing the complex to one or more wavelengths of light that interact with the one or more chemical groups; c) detecting a result from the interacting of one or more wavelengths of light that interact with the one or more chemical groups to provide an image of one or more single molecules. The image possesses a resolution better than 450 nm over an imaged area of at least 1×105 μm2, and wherein the image is obtained in a single detection step without variation of any detection settings.

In another method aspect, the present invention provides a method of imaging single molecules. The method comprises the steps of: a) exposing a test sample to a probe, wherein the probe comprises a first portion that specifically binds to a target molecule and a second portion that is modifiable to include one or more chemical groups that interact with light at one or more wavelengths, wherein the probe binds to a target molecule to provide a complex; b) modifying the second portion of the probe to include one or more of the chemical groups that interact with light; c) exposing the complex to one or more wavelengths of light that interact with the one or more chemical groups; d) detecting a result from the interacting of one or more wavelengths of light that interact with the one or more chemical groups to provide an image of one or more single molecules. The image possesses a resolution of better than 450 nm over an imaged area of at least 1×105 μm2, and wherein the image is obtained in a single detection step without variation of any detection settings.

### BRIEF DESCRIPTION OF THE FIGURES

FIG. 1 illustrates one embodiment of an SAO imaging device.

FIG. 2A illustrates another embodiment of an SAO imaging device.

FIG. 2B illustrates the internal structure of an illumination pattern generation module of an SAO imaging device, according to one embodiment.

FIG. 2C illustrates the internal structure of an illumination pattern generation module of an SAO imaging device, according to another embodiment.

FIG. 3 illustrates an SAO general method.

FIG. 4 shows a table of field of view diameters and area using an optical microscope having a field number of 26 mm.

FIG. 5 shows a table regarding the effect to the wavelength of light on resolution at a fixed numerical aperture (0.95).

FIG. 6 illustrates a method of imaging an mRNA or collections of mRNAs using a standard fluorescence microscope and a method of the present invention, which, in contrast, uses a system comprising an SAO imaging device to image the mRNA or collections of mRNAs.

FIG. 7 shows a portion of an SAO image of TOP1 mRNAs (bright/white/green dots) within an image area containing approximately 100 cells.

FIG. 8 shows the selection of a region of interest of an SAO image of TOP1 mRNAs, including a selection process graph based on spot intensity and quality.

FIG. 9 shows SAO images associated of HER2 mRNAs (bright/white dots) from an MCF7 human breast adenocarcinoma cell line. There is shown an image area containing over 100 cells, along with images of a section of the imaged are containing approximately 20 cells. With respect to the 20 cell image, on average each cell was shown to include around 72 copies of HER2 mRNA.

FIG. 10 shows two images associated with FKBP5 mRNAs (bright/white dots) from a A549 cells that were obtained using a standard fluorescent microscope (60×/1.41 NA0.1 oil). The image labeled “Minus Dex” shows cells prior to upregulation by the addition of 24 nM dexamethasone (approximately 13 cells); the image “Plus Dex” shows cells after addition of 24 nM dexamethasone for 8 hours (approximately 14 cells).

FIG. 11 shows two images ( 1/10 of full image) associated with FKBP5 mRNAs (bright/white dots) from a A549 cells that were obtained using a system comprising an SAO imaging device (20×). The image labeled “Minus Dex” shows cells prior to upregulation by the addition of 24 nM dexamethasone (over 50 cells); the image “Plus Dex” shows cells after addition of 24 nM dexamethasone for 8 hours (over 50 cells).

### DETAILED DESCRIPTION OF THE INVENTION

The present invention generally relates to imaging single molecules, or one or more collections of single molecules, and methods related to the imaging.

The method of imaging single molecules typically includes the steps of: 1) exposing a test sample (e.g., organism, exosome, tissue or cell) to a probe—where the probe includes a portion that specifically binds to a target molecule (e.g., RNA, protein, small molecule) and either a portion that is detectable as the result of one or more chemical groups that interact with light at one or more wavelengths or a portion that can be modified to include one or more chemical groups that interact with light at one or more wavelengths—which binds to a target molecule to provide a complex; 2) exposing the complex to one or more wavelengths of light that interact with the one or more chemical groups; 3) detecting a result from the interacting of one or more wavelengths of light that interact with the one or more chemical groups to provide an image of one or more single molecules, where the imaging system provides a detection resolution better than 450 nm over an imaged area of at least 1×10⁵ μm² in a single detection step (i.e., single set of data collected without variation of any detection settings (e.g., neither optics nor camera is moved)), thereby imaging a single molecule or a collection of single molecules. The imaging system is typically a system comprising a device that performs synthetic aperture optics (SAO imaging) or fluorescence polarization.

“SAO imaging” refers to an optical imaging method in which a series of patterned or structured light patterns are used to illuminate an imaging target in order to achieve resolution beyond what is set by physical constraints of the imaging apparatus, e.g., lens and camera. In SAO, an imaging target is selectively excited in order to detect spatial information on the target. Since there is a one-to-one relationship between the frequency (or Fourier) domain and the target domain, SAO can reconstruct the original imaging target by obtaining its spatial frequency information. See, U.S. patent application Ser. No. 12/728,110 filed Mar. 19, 2010, which is now U.S. Pat. No. 8,502,867, issued on Aug. 6, 2013, which is entitled, “Synthetic Aperture Optics Imaging Method Using Minimum Selective Excitation Patterns”, which is hereby incorporated-by-reference herein.

“Fluorescence Polarization” refers to the phenomenon where light emitted by a fluorophore has unequal intensities along different axes of polarization. For the microscopy applications discussed herein, Fluorescence Polarization uses polarizers in the path of the illuminating light and also before the imaging portion/camera of the apparatus. See, for example, Lokowicz, J. R., 2006. Principles of Fluorescence Spectroscopy (3rd ed., Springer, Chapter 10-12). Also see, Valeur, Bernard. 2001. Molecular Fluorescence: Principles and Applications Wiley-VCH, p. 29.

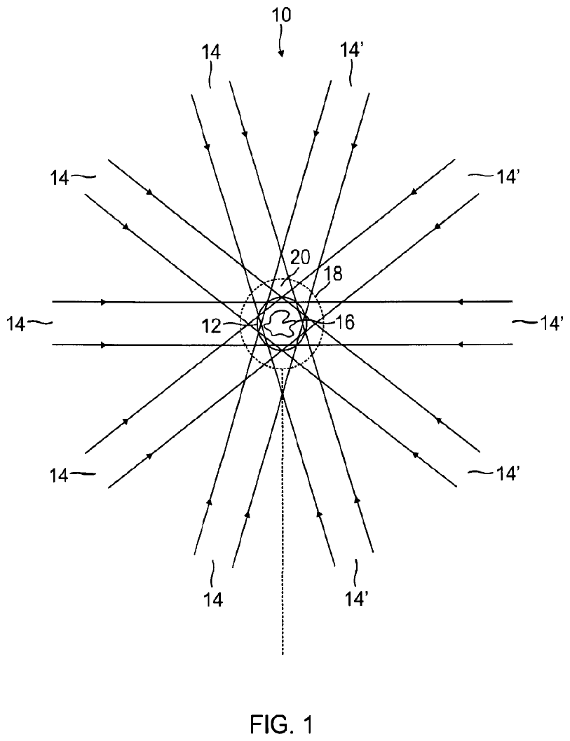

FIG. 1 illustrates one embodiment of an SAO imaging device. The device is a multiple beam pair optical scanner. The scanner is advantageous because it allows for parallel data acquisition which greatly enhances the acquisition speed of the scan. For scanners constructed with n beams, the degree of parallel data acquisition, and therefore, the degree of acquisition speed enhancement over known optical scanners, increases by an order of n squared. This assumes that the acquisition speed of the known optical scanners is limited by the speed of mechanical rotation of the sample or of a single beam pair.

The multiple beam pair optical scanner 10, in one embodiment, comprises an arc 12 of n source beams, generally 14, directed at a sample 16 where n is equal to ten and the arc 12 is a circle. Each of the n source beams 14 may have a different phase sequence or a different optical frequency. The phase sequence or frequency difference between each pair of the n source beams 14, 14′ is chosen to be unique among the phase sequence or frequency difference between the other pairs of the n source beams 14. The n source beams 14 overlap in a volume of space 20. A detector 18 detects a signal containing information from each of the multiple beam pairs within the arc 12 that is encoded with a unique phase sequence or carrier frequency which corresponds to the phase sequence or frequency difference of that pair.

The detector signal of the multiple beam pair optical scanner 10 using n source beams 14 passing through a volume of space 20 where the n beams 14 overlap and interact with the sample 16 can be calculated using methods known in the art. See, U.S. Pat. No. 6,016,196, which is incorporated-by reference herein.

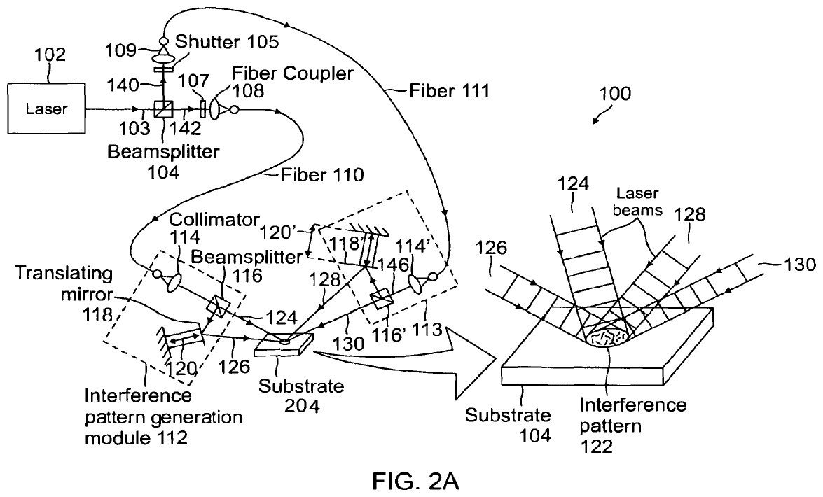

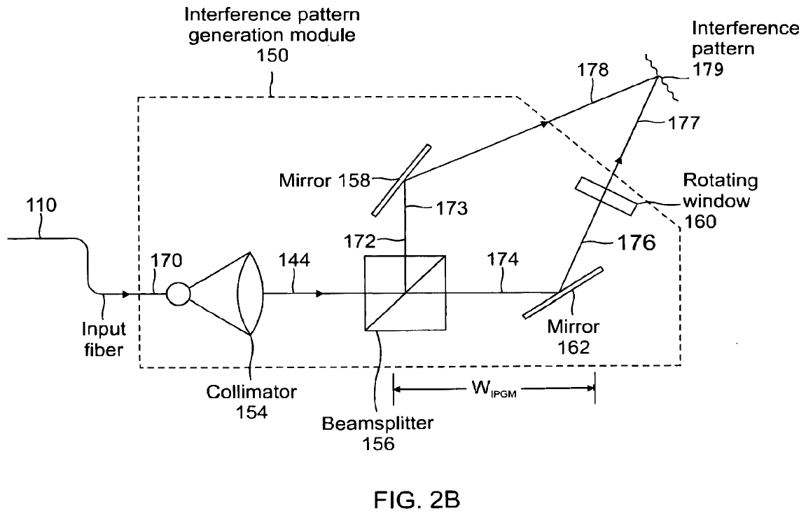

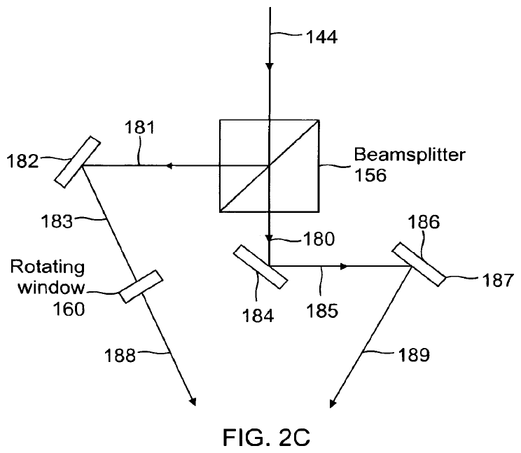

FIG. 2A illustrates an SAO imaging device (structured illumination apparatus) for selectively exciting the molecules, according to one embodiment. The illumination apparatus shown in FIG. 2A is merely exemplary, and various modifications may be made to the configuration of the illumination apparatus for SAO according to the present invention. The example illumination apparatus in FIG. 2A shows only two interference pattern generation modules (IPGM) 112, 113 for simplicity of illustration, but for certain applications there would be a larger number of IPGMs. Each IPGM is in modular form and is configured to generate one selective excitation pattern at a given pitch and orientation, corresponding to one conjugate pair of the k-space sampling points. Thus, there is a one-to-one relationship between an IPGM and a 2-D sinusoid selective excitation pattern at a given pitch and orientation and to one conjugate pair of the k-space sampling points. A larger number (N) of selective excitation patterns would require a larger number of IPGMs in the SAO illumination apparatus.

The structured illumination apparatus 100 generates multiple mutually-coherent laser beams, the interference of which produces interference patterns. Such interference patterns are projected onto the fixed cells substrate 204 and selectively excite cells and molecules under observation. Using the interference of multiple laser beams to generate the interference patterns is advantageous for many reasons. For example, this enables high-resolution excitation patterns with extremely large FOV (Field of View) and DOF (Depth of Field). Although the structured illumination apparatus of FIG. 2A is described herein with the example of generating excitation patterns for imaging molecules, it should be noted that the structured illumination apparatus of FIG. 2A can be used for any other type of application to generate excitation patterns for imaging any other type of target.

Referring to FIG. 2A, the structured illumination apparatus 100 includes a laser 102, a beam splitter 104, shutters 105, 107, fiber couplers 108, 109, a pair of optical fibers 110, 111 (one could alternatively use free beam architecture to deliver laser beams or any other suitable method) and a pair of interference pattern generation modules (IPGMs) 112, 113. As explained above, each IPGM 112, 113 generates an interference pattern (selective excitation pattern) that corresponds to one conjugate pair of k-space sampling points. The beam 103 of the laser 102 is split by the beam splitter 104 into two beams 140, 142. A pair of high-speed shutters 105, 107 is used to switch each beam 140, 142 “on” or “off” respectively, or to modulate the amplitude of each beam 140, 142, respectively. Such switched laser beams are coupled into a pair of polarization-maintaining optical fibers 111, 110 via fiber couplers 109, 108. Each fiber 111, 110 is connected to a corresponding interference pattern generation module 113, 112, respectively. The interference pattern generation module 113 includes a collimating lens 114′, a beam splitter 116′, and a translating mirror 118′, and likewise the interference pattern generation module 112 includes a collimating lens 114, a beam splitter 116, and a translating mirror 118.

The beam 144 from the optical fiber 110 is collimated by the collimating lens 114 and split into two beams 124, 126 by the beam splitter 116. The mirror 118 is translated by an actuator 120 to vary the optical path-length of the beam 126. Thus, an interference pattern 122 is generated on the substrate 204 in the region of overlap between the two laser beams 124, 126, with the phase of the pattern changed by varying the optical path-length of one of the beams 126 (i.e., by modulating the optical phase of the beam 126 by use of the translating mirror 118).

Similarly, the beam 146 from the optical fiber 111 is collimated by the collimating lens 114′ and split into two beams 128, 130 by the beam splitter 116′. The mirror 118′ is translated by an actuator 120′ to vary the optical path-length of the beam 128. Thus, the interference pattern 122 is generated on the substrate 204 in the region of overlap between the two laser beams 128, 130, with the pattern changed by varying the optical path-length of one of the beams 128 (i.e., by modulating the optical phase of the beam 128 by use of the translating mirror 118′).

As shown in FIG. 2A, each IPGM 112, 113 is implemented in modular form according to the embodiments herein, and one IPGM produces an interference pattern corresponding to one conjugate pair of k-space points. This modularized one-to-one relationship between the IPGM and the k-space points greatly simplifies the hardware design process for SAO according to the embodiments herein. As the number of selective excitation patterns used for SAO is increased or decreased, the SAO hardware is simply changed by increasing or decreasing the number of IPGMs in a modular manner. In contrast, conventional SAO apparatuses did not have discrete interference pattern generation modules but had a series of split beams producing as many multiple interferences as possible. Such conventional way of designing SAO apparatuses produced non-optimized or redundant patterns, slowing down and complicating the operation of the SAO system.

While this implementation illustrated in FIG. 2A is used for its simplicity, various other approaches can be used within the scope of the present invention. For example, the amplitude, polarization, direction, and wavelength, in addition to or instead of the optical amplitude and phase, of one or more of the beams 124, 126, 128, 130 can be modulated to change the excitation pattern 122. Also, the structured illumination can be simply translated with respect to the fixed cells to change the excitation pattern. Similarly, the fixed cells can be translated with respect to the structured illumination to change the excitation pattern. Also, various types of optical modulators can be used in addition to or instead of the translating mirrors 118, 118′, such as acousto-optic modulators, electro-optic modulators, a rotating window modulated by a galvanometer and micro-electro-mechanical systems (MEMS) modulators. In addition, although the structured illumination apparatus of FIG. 2A is described herein as using a laser 102 as the illumination source for coherent electro-magnetic radiation, other types of coherent electro-magnetic radiation sources such as an SLD (super-luminescent diode) may be used in place of the laser 102.

Also, although FIG. 2A illustrates use of four beams 124, 126, 128, 130 to generate the interference pattern 122, larger number of laser beams can be used by splitting the source laser beam into more than two beams. For example, 64 beams may be used to generate the interference pattern 122. In addition, the beam combinations do not need to be restricted to pair-wise combinations. For example, three beams 124, 126, 128, or three beams 124, 126, 130, or three beams 124, 128, 130, or three beams 126, 129, 130, or all four beams 124, 126, 128, 130 can be used to generate the interference pattern 122. Typically, a minimal set of beam combinations (two beams) is chosen as necessary to maximize speed. Also, the beams can be collimated, converging, or diverging. Although different from the specific implementations of FIG. 2A and for different applications, additional general background information on generating interference patterns using multiple beam pairs can be found in (i) U.S. Pat. No. 6,016,196, issued on Jan. 18, 2000 to Mermelstein, entitled “Multiple Beam Pair Optical Imaging,” (ii) U.S. Pat. No. 6,140,660, issued on Oct. 31, 2000 to Mermelstein, entitled “Optical Synthetic Aperture Array,” and (iii) U.S. Pat. No. 6,548,820, issued on Apr. 15, 2003 to Mermelstein, entitled “Optical Synthetic Aperture Array,” all of which are incorporated by reference herein.

FIG. 2B illustrates the internal structure of an illumination pattern generation module, according to one embodiment. The embodiment of FIG. 2B has a rotating window 160 in IPGM 150 that is placed after the mirror 162. The beam 170 from the optical fiber 110 is collimated by the collimating lens 154 and the collimated beam 144 is split into two beams 173, 174 by the beam splitter 156 (alternatively, a free beam architecture could be used to deliver the beam to the mirror). Beam 173 is reflected by mirror 158 and the reflected beam 178 is projected onto the imaging target to generate the interference pattern 179. Beam 174 is reflected by mirror 162 and the optical path-length of the reflected beam 176 is modulated by optical window 160 that is rotated, using a galvanometer, thereby modulating the optical phase of the corresponding beam 176 and generating a modulated beam 177. The interference pattern 179 is generated in the region of overlap between the two laser beams 177, 178, with the pattern changed by varying the optical path-length of one of the beams 177. By placing the rotating window 160 after the mirror 162, the width WIPGM and the size of IPGM 150 can be reduced, as compared to the embodiment of FIG. 2A and FIG. 2C illustrated below. Thus, the half-ring shaped structure holding the IPGMs can be made more compact, since the width WIPGM of the IPGM directly affects the radius of the half-ring, for example.

FIG. 2C illustrates the internal structure of an illumination pattern generation module, according to another embodiment. IPGMs in the embodiments of FIGS. 2A and 2B may produce two beams that do not have equal path length between the interfering point at the imaging target and the splitting point (i.e., the beam splitter). The non-equal path length may significantly reduce the sinusoidal contrast if a relatively short coherent-length laser is used and also limit the applicability of the SAO system to only a specific wavelength (e.g., 532 nm green laser) since only a small number of lasers with specific wavelengths have a sufficiently long coherent-length that can be used with such non-equal-path IPGMs for good sinusoidal contrast. Compared to the embodiment of FIG. 2A, the embodiment of FIG. 2C uses additional folding mirrors to achieve equal paths between the two split beams. The laser beam 144 is split into beams 181, 180 by beam splitter 156. Beam 181 is reflected by mirror 182 and its optical path-length is modulated by rotating window 160 to generate beam 188. On the other hand, beam 180 is reflected twice by two mirrors 184, 187 to generate the reflected beam 189. Beam 188 and 189 eventually interfere at the imaging target to generate the selective excitation patterns. By use of two mirrors 184, 186, the optical path 144-180-185-189 is configured to have a length substantially equal to the length of the optical path 181-183-188. This equal-path scheme allows lasers with short coherent lengths to be used to generate interference patterns with high contrast. Moreover, this equal-path scheme enables the SAO system to be used with wavelengths other than 532 nm, thus making multiple-color SAO practical.

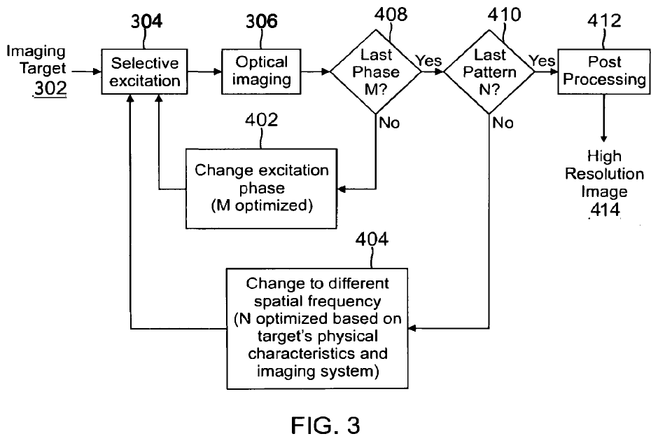

FIG. 3 illustrates one SAO general method. Selective excitation (or illumination) 304 is applied to an imaging target 302, and the light scattered or fluoresced from the imaging target 302 is captured by optical imaging 306. Selective excitation 304 is applied to the imaging target 302 by an illumination apparatus that is configured to cause interference of two light beams on the imaging target 302. The excited target 302 emits signals (or photons), and the emitted signals are captured in an optical imaging system including an objective lens and an imaging sensor (or imager). It is determined 408 whether the images corresponding to all M phases of the 2D sinusoid excitation pattern were obtained.

If images corresponding to all the phases of the 2D sinusoid excitation pattern were not obtained in step 408, the excitation phase is changed 402 and steps 304, 306, 408 are repeated for the changed excitation phase. If images corresponding to all the phases of the 2D sinusoid excitation pattern were obtained in step 408, then it is determined 410 whether the images corresponding to all the 2D sinusoid excitation patterns were obtained. If images corresponding to all the 2D sinusoid excitation patterns were not obtained in step 410, the excitation pattern is changed by using a different spatial frequency (e.g., changing the pitch and orientation ϕ of the 2D sinusoid pattern) and steps 304, 306, 408, 402, 410, 404 are repeated for the next selective excitation pattern. If images corresponding to all the 2D sinusoid excitation patterns were obtained in step 410, then the captured images are sent to a computer for SAO post processing 412 and visualization to obtain the high-resolution images 414 of the imaging target 302 from the captured lower resolution raw images. As explained above, the raw images captured by optical imaging 306 have a resolution insufficient to resolve the objects on the imaging target 302, while the high resolution image 414 reconstructed by SAO post-processing 412 has a resolution sufficient to resolve the objects on the imaging target 302.

“Resolution” refers to the shortest distance between two points in a test sample/specimen that can be distinguished by an observer or imaging system as two separate entities. There are several equations that have been derived with respect to resolution of an optical microscope to express the relationship between numerical aperture, wavelength, and resolution:

Resolution (r)=λ/(2NA) (1)

Resolution (r)=0.61λ/NA (2)

Resolution (r)=1.22λ/(NA(obj)+NA(cond)) (3)

where “r” is resolution (the smallest resolvable distance between two objects), “NA” is a general term for the microscope numerical aperture, “λ” is the imaging wavelength, “NA(obj)” equals the objective numerical aperture, and “NA(cond)” is the condenser numerical aperture.

“Numerical aperture” of a microscope objective is a measure of its ability to gather light and resolve fine specimen detail at a fixed object distance.

“Field of view” is the diameter of the view field expressed in millimeters measured at the intermediate plane in an optical microscope. The “field-of-view number”, or “field number”, is expressed in millimeters and when divided by magnification provides the actual FOV.

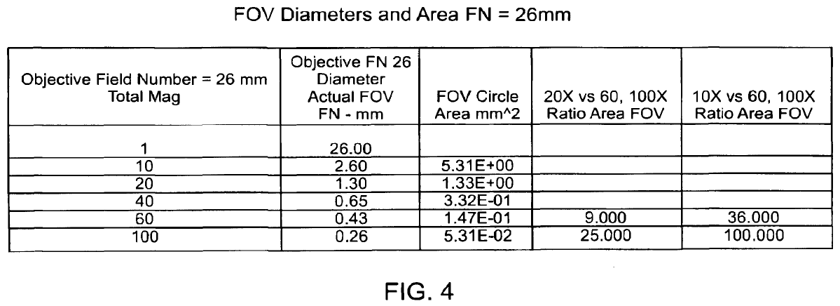

FIG. 4 shows a table of field of view diameters and area using an optical microscope having a field number of 26 mm.

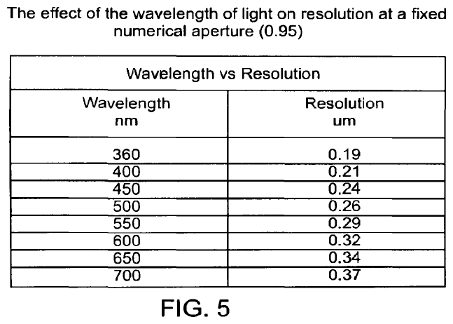

FIG. 5 shows a table regarding the effect to the wavelength of light on resolution at a fixed numerical aperture (0.95).

Nonlimiting examples of molecules that are imaged using the method of the present invention include: messenger ribonucleic acids (mRNAs); long non-coding ribonucleic acids (Inc RNAs); small nuclear ribonucleic acids (snRNAs); subgenomic ribonucleic acids (sgRNA); viral RNA; small interfering RNA (siRNA); non-coding RNA (e.g., tRNA and rRNA); transfer messenger RNA (tmRNA); micro RNA (miRNA); piwi-interacting rNA (piRNA); small nucleolar RNA (snoRNA); antisense RNA; double-stranded RNA (dsRNA); heterogeneous nuclear RNA (hnRNA); chromosomes (e.g., through chromosomal painting); double- and single-stranded deoxyribonucleotides (DNA); BrdU or EdU incorporated into replicated DNA strands of proliferating cells; proteins; glycans; small biological and non-biological molecules.

The portion of a probe that specifically binds to a target molecule is typically: a DNA or RNA molecule (e.g., antisense oligomer or polymer); a DNA or RNA analog (e.g., inclusion of non-natural nucleotides); an antibody; or an aptamer. The detectable portion of a probe is usually a fluorescent group. Nonlimiting examples of such fluorescent groups include:

fluorescent organic dyes such as xanthenes (e.g., fluoresceins, rhodamines, etc.), cyanines, luminescent groups (e.g., lanthanides, chelates, ruthenium, etc.), coumarins, pyrenes, bodipy dyes, and FLAsh; non-organic chromophores such as semiconductor nanocrystals (quantum dots), silicon, gold, and metal nanoparticles; intercalator dyes such as DAPI, DRAQ-5, and Hoechst 33342; expressible fluorescent proteins such as Green Fluorescent Protein (GFP), yellow FP, red FP, etc.

Nonlimiting examples of DNA or RNA analogs include those that possess the following: spermine tails; MGB; LNA; PNA; RNA 2′ modified sugars; amidate backbone; morpholino backbone; thioate backbone; and, TSQ dye modulators.

Nonlimiting examples of fluorescence dye labeled nucleic acid probe types include: Singer probes (multilabeled); Stellaris probes (single labeled); DOPE-FISH probes (double labeled); MTRTP probes; fluorescent labeled BAC probes; FRET-Quenched probes (e.g., Molecular Beacons, linear F-Q Probes, Hyb probes); ECHO probes; Dye labeled dendrimers; triggered fluorescence (e.g., Kool probes, ligation activated); Caged probes (e.g., photo triggered FI); profluorescent dyes (e.g., chemically activated—oxidative, reductive, acid, base, etc.).

The detection of the probe/target molecule complex typically involves the generation of a fluorescent signal one light wavelength from the probe after absorption of a different light wavelength from a source. Nonlimiting examples of fluorescent signal generation include aptamer quenching and enzyme generated fluorescence. The signal can also be generated or amplified using various techniques, including, but not limited to: hybridization capture; rolling circle amplification; B-DNA; polymerase chain reaction; and, enzyme generated fluorescence.

Where a probe is modified to include the chemical compound that interacts with light, any suitable process known in the art of chemical conjugation can be used. Nonlimiting examples of such processes include: Solution or Solid Phase oligo synthesis via phosphoramidite, phosphonate ester, triester intermediates, or the like; click chemistry (copper catalyzed and copper free); Diels-Alder reaction; Staudinger ligation; hydrazone ligation; oxime ligation; native chemical ligation; tetrazine ligation; maleimide-thiol ligation; active ester-amine ligation; carbodiimide (EDC) phosphate or carboxy conjugation.

In one aspect, the method is used to image an mRNA or collections of mRNAs. This method typically includes the steps of: 1) obtaining a large number of oligonucleotides that are capable of hybridizing to one or more mRNA targets, where each oligonucleotide includes a single fluorescent label, to provide a set of singly-labeled oligonucleotides; 2) obtaining a sample preparation (e.g., a preparation including a number of live cells); 3) allowing the set of singly-labeled oligonucleotides to interact with the sample preparation such that a substantial number of the singly-labeled oligonucleotides hybridize to one or more mRNA targets within the cells, to afford a set of oligonucleotide-mRNA hybridized products; 4) detecting the set of oligonucleotide-mRNA hybridized products by imaging them using an imaging system, such as an imaging system comprising a device that performs synthetic aperture optics (SAO imaging) or fluorescence polarization, that provides resolution of better than 450 nm over an imaged area of at least 1×10⁵ μm² in a single detection step (i.e., single set of data collected without variation of any detection settings (e.g., neither optics nor camera is moved)).

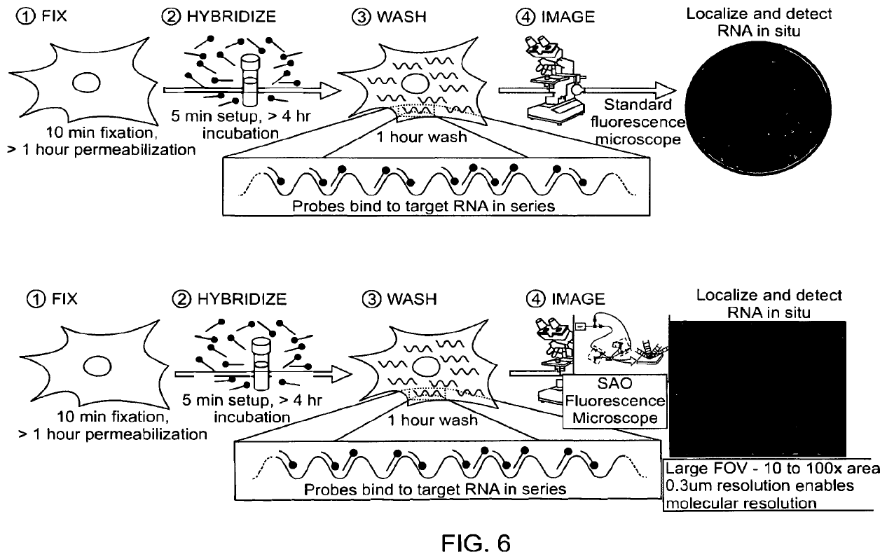

FIG. 6 illustrates a method of imaging an mRNA or collections of mRNAs using a standard fluorescence microscope and a method of the present invention, which, in contrast, uses an imaging system comprising a device that performs synthetic aperture optics (SAO imaging) to image the mRNA or collections of mRNAs.

The large number of oligonucleotides used in the method to construct probes typically includes at least 30 different oligonucleotides. Oftentimes, 40 to 60 oligonucleotides are used, with 48 being commonly employed. The number of nucleotides included in the oligonucleotides is usually between 15 and 40. Oligonucleotides containing 15-20, 17-22 or 17-25 are oftentimes used.

Oligonucleotides of the probes are typically designed using a suitable software package, such as Probe Designer. See www.singlemoleculefish.com. The oligonucleotides can be synthesized by any appropriate method, including solid phase synthesis using an automated DNA/RNA synthesizer. Attachment of a fluorescent label to the oligonucleotides, thereby providing probes, is usually performed by pooling the oligonucleotides and coupling each to a single fluorophore in the same reaction.

In another aspect, the method is used to image an Inc RNA or collections of Inc RNAs. This method typically includes the steps of: 1) obtaining one or more oligonucleotides that are capable of hybridizing to one or more Inc RNA targets, where each oligonucleotide includes one or more fluorescent labels, to provide one or more Inc RNA probes; 2) obtaining a sample preparation (e.g., a preparation including a number of live cells); 3) allowing the one or more Inc RNA probes to interact with the sample preparation such that a substantial number of the probes hybridize to one or more Inc RNA targets within the cells, to afford a set of probe-Inc RNA hybridized products; 4) detecting the set of probe-Inc RNA hybridized products by imaging them using an imaging system, such as a system comprising a device that performs synthetic aperture optics (SAO imaging) or fluorescence polarization, that provides resolution of better than 450 nm over an imaged area of at least 1×10⁵ μm² in a single detection step (i.e., single set of data collected without variation of any detection settings).

In another aspect, the method is used to image an snRNA or collections of snRNAs. This method typically includes the steps of: 1) obtaining one or more oligonucleotides that are capable of hybridizing to one or more snRNA targets, where each oligonucleotide includes one or more fluorescent labels, to provide one or more snRNA probes; 2) obtaining a sample preparation (e.g., a preparation including a number of live cells); 3) allowing the one or more snRNA probes to interact with the sample preparation such that a substantial number of the probes hybridize to one or more snRNA targets within the cells, to afford a set of probe-snRNA hybridized products; 4) detecting the set of probe-snRNA hybridized products by imaging them using an imaging system, such as a system comprising a device that performs synthetic aperture optics (SAO imaging) or fluorescence polarization, that provides resolution of better than 450 nm over an imaged area of at least 1×10⁵ μm² in a single detection step (i.e., single set of data collected without variation of any detection settings).

In another aspect, the method is used to image all, or a portion of, a chromosome. This method typically includes the steps of: 1) obtaining one or more oligonucleotides that are capable of hybridizing to one or more locations within a target chromosome, where each oligonucleotide includes one or more fluorescent labels, to provide one or more chromosomal probes; 2) obtaining a sample preparation (e.g., a preparation including a number of live cells); 3) allowing the one or more chromosomal probes to interact with the sample preparation such that a substantial number of the probes hybridize to one or more locations within the chromosomal target within the cells, to afford a set of probe-chromosome hybridized products; 4) detecting the set of probe-chromosome hybridized products by imaging them using an imaging system, such as a system comprising a device that performs synthetic aperture optics (SAO imaging) or fluorescence polarization, that provides resolution of better than 450 nm over an imaged area of at least 1×10⁵ μm² in a single detection step (i.e., single set of data collected without variation of any detection settings).

In another aspect, the method is used to image cell proliferation using the incorporation of BrdU into a replicating DNA strand of the cell. This method typically includes the steps of: 1) obtaining a sample preparation (e.g., a preparation including a number of live cells); 2) providing an amount of BrdU to the sample preparation and incubating the provided BrdU with the sample preparation for a time period that allows for a significant amount of the BrdU to be incorporated into proliferating cells; 3) providing an amount of an anti-BrdU antibody comprising one or more fluorescent groups to the sample preparation and incubating the provided antibody with the sample preparation for a time period that allows for binding of a significant amount of the antibody to the BrdU incorporated into the replicated DNA; 4) detecting the BrdU bound antibodies by imaging them using an imaging system, such as a system comprising a device that performs synthetic aperture optics (SAO imaging) or fluorescence polarization, that provides resolution of better than 450 nm over an imaged area of at least 1×10⁵ μm² in a single detection step (i.e., single set of data collected without variation of any detection settings).

In another aspect, the method is used to image cell proliferation using the incorporation of EdU into a replicating DNA strand of the cell. This method typically includes the steps of: 1) obtaining a sample preparation (e.g., a preparation including a number of live cells); 2) providing an amount of EdU to the sample preparation and incubating the provided EdU with the sample preparation for a time period that allows for a significant amount of the EdU to be incorporated into proliferating cells; 3) providing an amount of a fluorescent-labeled, azide-based Click reagent under conditions that allow reaction between the incorporated EdU and the Click reagent; 4) detecting the EdU-Click reagent reaction products by imaging them using an imaging system, such as a system comprising a device that performs synthetic aperture optics (SAO imaging) or fluorescence polarization, that provides resolution better than 450 nm over an imaged area of at least 1×10⁵ μm² in a single detection step (i.e., single set of data collected without variation of any detection settings).

The method of the present invention provides a means of quantitating individual molecules (e.g., mRNAs, Inc RNAs, snRNAs, chromosomes, DNA strands including BrdU or EdU, proteins, glycans, small molecules) within the cytoplasm and nucleus of cells. Images of individual molecules are resolved at resolutions better than 450 nm, 400 nm, 350 nm, 300 nm, or 250 nm. Oftentimes individual molecules are resolved at resolutions better than 200 nm, 150 nm or 100 nm. Those resolutions are achievable over an imaged area of at least 1×10⁵ μm² in a single detection step. In certain cases the resolution applies to an imaged area of at least 1×10⁶ m², 5×10⁶ μm², 1×10⁷ μm² or 5×10⁷ μm². These areas correspond to a field of 100s to 1000s of cells.

The method of the present invention does not require extremely high molecular densities of one or more fluorophores to achieve high resolution over a large field of view area. For instance, images of individual molecules are resolved at resolutions better than 450 nm, 400 nm, 350 nm, 300 nm, 250 nm, 200 nm, 150 nm or 100 nm in an imaged area of at least 1×10⁵ μm², 1×10⁶ μm², 5×10⁶ μm², 1×10⁷ μm², or 5×10⁷ μm² in a single detection step, even where the density of fluorophores in the field of view area is less than 10,000 molecules per m². Typically, that resolution is achieved even where the density of fluorophores in the field is less than 1000 molecules per m², 100 molecules per m² or 10 molecules per m².

Over the areas discussed above, the method is typically able to detect at least 1×10² distinct molecular complexes, where the complexes comprise at least one probe bound to a target molecule, in a single detection step. In certain cases, the method can detect at least 1×10³, 1×10⁴, 1×10¹, 1×10⁶, 1×10⁷, 1×10⁸ or 1×10⁹ distinct molecular complexes in a single detection step.

Also related to the areas discussed above, the method is typically able to detect/image greater than 20 cells (SAO image at standard 20× objective) in a single detection step. In certain cases, the method is able to detect/image greater than 50, 100, 150, 200, 250, or 300 cells in a single detection step.

Another advantage of the method is the long working distance of the instrument objective, which makes it possible to obtain high resolution images of areas restrictive (mechanically) with respect to Standard 60× or 100× immersion lenses. The long working distance, along with the method's large Depth of Field enable focusing through thick substrates to image a desired area. The method can, for example, obtain images through samples greater than 0.1 mm thick (e.g., plastic samples (COP). In certain cases, the method can obtain images through samples greater than 0.25 mm thick, 0.50 mm thick, 0.75 mm thick or 1.0 mm thick.

The quantification afforded by the method of the present invention includes several different aspects. One can quantify gene expression across an entire sample of cells, within different cells of the sample, and within different regions of each cell of the sample. One can quantify particular gene variations (e.g., SNPs) or mutations within the same cell or different cells. One can also quantify the following: multi-locus gene synthesis; translocation of genetic elements; and, the rate of cell proliferation.

In certain cases, more than one type of probe is used at the same time in the method (i.e., multiplexing). The probe types are different with respect to both specific binding portions and chemical detection portions. As a nonlimiting example, more than one set of singly-labeled oligonucleotides can be used in a method for detection of single mRNAs, where each set has a different fluorophore as its label. The use of different mRNA targets allows one to simultaneously quantify and compare the expression of two, three, four or more genes.

Quantification provided by the method of the present invention furthermore extends beyond quantifying the number of molecular complexes within a cellular region; the method provides for quantification of distance between molecular complexes or between regions of a chromosome that are complexed to different probes where multiplexing is employed. One can measure a distance between to complexes equal to or less than 450 nm, 400 nm, 350 nm, 300 nm, 250 nm, 200 nm, 150 nm or 100 nm. Using this method of measurement one is able, for example, to quantify the distance between locations on a single chromosome or the distance between regions of different chromosomes. These types of measurements can elucidate chromosomal “cross talk”, i.e., how different chromosomal regions affect one another with respect to functional activity such as gene expression.

As discussed above, the method of the present invention can be used to obtain several different types of information regarding genes (e.g., expression levels). Nonlimiting examples of genes that are examined using the method include: ABL1; ABL2; ACSL3; AF15Q14; AF1Q; AF3p21; AF5q31; AKAP9; AKT1; AKT2; ALDH2; ALK; ALO17; APC; ARHGEF12; ARHH; ARID1A; ARID2; ARNT; ASPSCR1; ASXL1; ATF1; ATIC; ATM; ATRX; BAP1; BCL10; BCL11 A; BCL11B; BCL2; BCL3; BCL5; BCL6; BCL7A; BCL9; BCOR; BCR; BHD; BIRC3; BLM; BMPR1A; BRAF; BRCA1; BRCA2; BRD3; BRD4; BRIP1; BTG1; BUB1B; C12orf9; C15orf21; C15orf55; C16orf75; C2orf44; CAMTA1; CANT1; CARD11; CARS; CBFA2T1; CBFA2T3; CBFB; CBL; CBLB; CBLC; CCDC6; CCNB1IP1; CCND1; CCND2; CCND3; CCNE1; CD273; CD274; CD74; CD79A; CD79B; CDH1; CDH11; CDK12; CDK4; CDK6; CDKN2A; CDKN2a(p14); CDKN2C; CDX2; CEBPA; CEP1; CHCHD7; CHEK2; CHIC2; CHN1; CIC; CIITA; CLTC; CLTCL1; CMKOR1; COL1A1; COPEB; COX6C; CREB1; CREB3L1; CREB3L2; CREBBP; CRLF2; CRTC3; CTNNB1; CYLD; D10S170; DAXX; DDB2; DDIT3; DDX10; DDX5; DDX6; DEK; DICER1; DNM2; DNMT3A; DUX4; EBF1; ECT2L; EGFR; EIF4A2; ELF4; ELK4; ELKS; ELL; ELN; EML4; EP300; EPS 15; ERBB2; ERCC2; ERCC3; ERCC4; ERCC5; ERG; ETV1; ETV4; ETV5; ETV6; EVI1; EWSR1; EXT1; EXT2; EZH2; EZR; FACL6; FAM22A; FAM22B; FAM46C; FANCA; FANCC; FANCD2; FANCE; FANCF; FANCG; FBXO11; FBXW7; FCGR2B; FEV; FGFR1; FGFRIOP; FGFR2; FGFR3; FH; FHIT; FIP1L1; FLU; FLJ27352; FLT3; FNBP1; FOXL2; FOXO1A; FOXO3A; FOXP1; FSTL3; FUBP1; FUS; FVT1; GAS7; GATA1; GATA2; GATA3; GMPS; GNA11; GNAQ; GNAS; GOLGA5; GOPC; GPC3; GPHN; GRAF; H3F3A; HCMOGT-1; HEAB; HERPUD1; HEY1; HIP 1; HIST1H4I; HLF; HLXB9; HMGA1; HMGA2; HNRNPA2B1; HOOK3; HOXA11; HOXA13; HOXA9; HOXC11; HOXC13; HOXD11; HOXD13; HRAS; HRPT2; HSPCA; HSPCB; IDH1; IDH2; IGH@; IGK@; IGL@; IKZF1; IL2; IL21R; IL6ST; IL7R; IRF4; IRTA1; ITK; JAK1; JAK2; JAK3; JAZF1; JUN; KDM5A; KDM5C; KDM6A; KDR; KIAA1549; KIF5B; KIT; KLK2; KRAS; KTN1; LAF4; LASP1; LCK; LCP1; LCX; LHFP; LIFR; LMO1; LMO2; LPP; LRIG3; LYL1; MADH4; MAF; MAFB; MALT1; MAML2; MAP2K4; MDM2; MDM4; MDS1; MDS2; MECT1; MED 12; MEN1; MET; MITF; MKL1; MLF1; MLH1; MLL; MLL2; MLL3; MLLT1; MLLT10; MLLT2; MLLT3; MLLT4; MLLT6; MLLT7; MN1; MPL; MSF; MSH2; MSH6; MSI2; MSN; MTCP1; MUC1; MUTYH; MYB; MYC; MYCL1; MYCN; MYD88; MYH11; MYH9; MYST4; NACA; NBS1; NCOA1; NCOA2; NCOA4; NDRG1; NF1; NF2; NFE2L2; NFIB; NFKB2; NIN; NKX2-1; NONO; NOTCH1; NOTCH2; NPM1; NR4A3; NRAS; NSD1; NTRK3; NUMA1; NUP214; NUP98; OLIG2; OMD; P2RY8; PAFAH1B2; PALB2; PAX3; PAX5; PAX7; PAX8; PBRM1; PBX1; PCM1; PCSK7; PDE4DIP; PDGFB; PDGFRA; PDGFRB; PER1; PHF6; PHOX2B; PICALM; PIK3CA; PIK3R1; PIM1; PLAG1; PML; PMS1; PMS2; PMX1; PNUTL1; POU2AF1; POU5F1; PPARG; PPP2R1A; PRCC; PRDM1; PRDM16; PRF1; PRKAR1A; PRO1073; PSIP2; PTCH; PTEN; PTPN11; RAB5EP; RAS5IL1; RAF1; RALGDS; RANBP17; RAP1GDS1; RARA; RB1; RBM15; RECQL4; REL; RET; ROS1; RPL22; RPN1; RUNDC2A; RUNX1; RUNXBP2; SBDS; SDC4; SDH5; SDHB; SDHC; SDHD; SEPT6; SET; SETD2; SF3B1; SFPQ; SFRS3; SH3GL1; SIL; SLC34A2; SLC45A3; SMARCA4; SMARCB1; SMO; SOCS1; SOX2; SRGAP3; SRSF2; SS18; SS18L1; SSH3BP1; SSX1; SSX2; SSX4; STK11; STL; SUFU; SUZ12; SYK; TAF15; TAL1; TAL2; TCEA1; TCF1; TCF12; TCF3; TCF7L2; TCL1A; TCL6; TET2; TFE3; TFEB; TFG; TFPT; TFRC; THRAP3; TIF1; TLX1; TLX3; TMPRSS2; TNFAIP3; TNFRSF14; TNFRSF17; TNFRSF6; TOP1; TP53; TPM3; TPM4; TPR; TRA@; TRB@; TRD@; TRIM27; TRIM33; TRIP11; TSC1; TSC2; TSHR; TTL; U2AF1; USP6; VHL; VTI1A; WAS; WHSC1; WHSC1L1; WIF1; WRN; WT1; WTX; WWTR1; XPA; XPC; XPO1; YWHAE; ZNF145; ZNF198; ZNF278; ZNF331; ZNF384; ZNF521; ZNF9; ZRSR2.

Where the target molecule of the method is mRNA, nonlimiting examples of targeted mRNAs include: CCNB1 mRNA, CENPE mRNA, AURKB mRNA, PLK1 mRNA, PLK4 mRNA, TAGLN mRNA, ACTG2 mRNA, TPM1 mRNA, MYH111 mRNA, DES mRNA, EIF1AX mRNA, AR mRNA, HSPD1 mRNA, HSPCA mRNA, K-ALPHA1 mRNA, MLL5 mRNA, UGT2B15 mRNA, WNT5B5 mRNA, ANXA11 mRNA, FOS mRNA, SFRP1 mRNA, FN1 mRNA, ITGB8 mRNA, THBS2 mRNA, HNT mRNA, CDH10 mRNA, BMP4 mRNA, ANKH mRNA, SEP4 mRNA, SEP7 mRNA, PTN mRNA, VEGF mRNA, SRY mRNA, EGR3 mRNA, FoxP1 mRNA, FoxM1 mRNA, TGCT1 mRNA, ITPKB mRNA, RGS4 mRNA, and BACE1 mRNA.

In certain cases, methods of the present invention, and related kits, are used for the in vivo, in vitro, and/or in situ analysis of nucleic acids, proteins, antibodies or haptens. Such nucleic acids include, without limitation, genomic DNA, chromosomes, chromosome fragments and genes (DNA-FISH). Nonlimiting examples of methods by which the nucleic acids or proteins are analyzed include: PCR; in situ PCR; flow cytometry; fluorescence microscopy; chemiluminescence; immunohistochemistry; virtual karyotype; gene assay; DNA microarray (e.g., array comparative genomic hybridization (array CGH)); gene expression profiling; Gene ID; Tiling array; immunofluorescence; FISSEQ (Fluorescence in Situ sequencing); and, in situ hybridizations such as FISH, SISH, and CISH.

In certain other cases, methods of the present invention, and related kits, are used for the in vivo, in vitro or in situ analysis of nucleic acids for chromosomal aberrations. Nonlimiting examples of such aberrations include: aneuploidy; potential breakpoint; insertion; inversion; deletion; duplication; gene amplification; rearrangement; and translocation. Such aberrations are oftentimes associated with a normal condition or a disease (e.g., congenital disease, cancer or infection).

Test samples for the method may be obtained from any suitable source, including, without limitation, human, animal or plant sources. The samples typically include cells and may be removed from the sample source (in vitro) or retained in the source (in vivo). For example, the samples may be derived from tissue biopsy, blood, urine, fecal matter, saliva and sweat. In certain cases, the sample is fixed to a sample substrate (e.g., slide, flow cell, microplate).

The method of the present invention are used in the diagnosis, monitoring and/or prognosis of diseases or other conditions. For instance, one can diagnose a particular disease (e.g., breast cancer; colon cancer; prostate cancer; testicular cancer; infection; and, Alzheimer's disease) by assessing the activity of one or more specific genes within a tissue sample.

In one, nonlimiting case, the present invention provides a method of diagnosing a congenital disorder, cancer, or infection associated with a chromosomal aberration. The method comprises the steps of: obtaining a tissue, exosome or cell sample from a subject, where the tissue sample comprises a nucleic acid sequence; determining whether a chromosomal aberration is present in the nucleic acid sequence; and, diagnosing the congenital genetic disorder, cancer, or infection if the chromosomal aberration is present in the tissue, exosome or cell sample. The tissue, exosome or cell sample is typically mammalian (e.g., human) in origin.

Regarding disease diagnosis, the method can diagnose the diseases discussed at the following sites (which are herein incorporated by reference for all purposes):

http://www.cdc.gov/diseasesconditions/az/a.html;

http://www.medicinenet.com/diseases_-and_conditions/alpha_a.htm;
http://en.wikipedia.org/wiki/Lists_of_diseases; and,

http://www.rightdiagnosis.eom/lists/#undefined.

Nonlimiting examples of cancer types that can be diagnosed by the method of the present invention include: Bladder Cancer; Breast Cancer; Colon Cancer; Rectal Cancer; Endometrial Cancer; Kidney (Renal and Cell) Cancer; Leukemia; Lung Cancer; Melanoma; non-Hodgkin Lymphoma; Pancreatic Cancer; Prostate Cancer; and Thyroid Cancer.

Nonlimiting examples of virus-based diseases that can be diagnosed by the method of the present invention include: Avian Influenza (Flu); HIV/AIDS; Hepatitis A; Hepatitis B; Hepatitis C; H1N1 Influenza (Swine flu); Adenovirus Infection; Respiratory Syncytial Disease; Rhinovirus Infection; Herpes Simplex; Chicken Pox (Varicella); Measles (Rubeola); German Measles (Rubella); Mumps (Epidemic Protitis); Small Pox (Variola); Warts Kawasaki Disease; Yellow Fever; Dengue Fever; Viral Gastroenteritis; Viral Fevers; Cytomegalovirus Disease; Rabies; Polio; Slow Virus Disease; and, Arboviral Enephalitis. Nonlimiting examples of viruses that can be detected/diagnosed with respect to the preceding diseases include: Adenovirus; Coxsackievirus; Epstein-Barr Virus; Hepatitis A Virus; Hepatitis B Virus; Hepatitis C Virus; Herpes Simplex Virus, Type 1; Herpes Simplex Virus, Type 2; Cytomegalovirus; Human Herpesvirus, Type 8; HIV; Influenza Virus; Measles Virus; Mumps Virus; Human Papillomavirus; Parainfluenza Virus; Polio Virus; Respiratory Synctial Virus; Rubella Virus; and, Varicella-Zoster Virus.

Nonlimiting examples of parasitic diseases that can be diagnosed using the method of the present invention include (independent of host—e.g., dog, worms, birds, plant, animal, human): Acanthamoeba Keratitis; Amoebiasis (Entamoeba Histolytica and Others); Ascariasis (Ascaris Lumbricoides); Babesiosis; Baylisascariasis; Chagas Disease (Trypanosoma Cruzii); Clonorchiasis; Cochliomyia; Cryptosporidiosis; Diphyllobothriasis; Dracunculiasis (caused by the Guinea Worm); Echinococcosis; Elephantiasis; Enterobiasis; Fascioliasis; Fasciolopsiasis; Filariasis; Giardiasis; Gnathostomiasis; Hymenolepiasis; Hookworm; Isosporiasis; Katayama Fever; Leishmaniasis; Malaria (Plasmodium Falciparum, P. Vivax, P. Malariae, P. Ovale, and P. Knowlesii); Metagonimiasis; Myiasis; Onchocerciasis; Pediculosis; Scabies; Schistosomiasis; Sleeping Sickness; Strongyloidiasis; Taeniasis (cause of Cysticercosis); Toxocariasis; Toxoplasmosis (Toxoplasma Gondii); Trichinosis; and, Trichuriasis. Nonlimiting examples of related pathogens that can be detected using the method include: Acanthamoeba; Anisakis; Ascaris Lumbricoides; Botfly; Balantidium Coli; Bedbug; Cestoda (Tapeworm); Chiggers; Cochliomyia Hominivorax; Entamoeba Histolytica; Fasciola Hepatica; Giardia Lamblia; Hookworm; Leishmania; Linguatula Serrata; Liver Fluke; Loa Loa; Paragonimus—Lung Fluke; Pinworm; Plasmodium Falciparum; Schistosoma; Strongyloides Stercoralis; Mite; Tapeworm; Toxoplasma Gondii; Trypanosoma; Whipworm; and, Wuchereria Bancrofti.

Nonlimiting example of bacteria that can be detected using the method of the present invention include: Acinetobacter; Anthrax; Campylobacter; Gonorrhea; Group B Streptococcus; Klebsiella Pneumoniae; Methicillin-resistant Staphylococcus Aureus (MRSA); Neisseria Meningitis; Salmonella, Non-Typhoidal Serotypes; Shigella; Streptococcus Pneumoniae; Tuberculosis; Typhoid Fever; Vancomycin-Resistant Enterococci (VRE); Vancomycin-Intermediate/Resistant Staphylococcus Aureus (VISA/VRSA).

In another, nonlimiting case, the method of the present invention, and related kits, are used for detection of changes in RNA expression levels—e.g., mRNA and its complementary DNA (cDNA). The compositions may be used on in vitro, in vivo, or in situ samples (e.g., mammalian samples, such as human samples). Such samples include, without limitation, the following: bone marrow smears; blood smears; paraffin embedded tissue preparations; enzymatically dissociated tissue samples; bone marrow; amniocytes; cytospin preparations; and, imprints.

In another, nonlimiting case, the tissue sample is fixed and permeabilized and probed with target RNA specific, singly labeled probes associated with the disease and subjected to SAO imaging having 450 nm resolution or better (e.g., 300 nm or 150 nm) over an imaged area of at least 1×10⁶ μm².

Prognostic assays (companion diagnostics) can also be run using the method of the present invention. For example, one can use FISH or modified FISH techniques to detect rearrangements of the ERG and ETV1 genes and measure a loss of the PTEN gene. One can use the degree of ERG/ETV1 genetic aberrations in the presence or absence of the PTEN gene as an indicator that chemotherapy will or will not be successful for prostate cancer patients. Other, nonlimiting examples of companion diagnostic methods in which one uses the method of the present invention include: BRACAnalysis to identify patients who are more likely to respond to therapeutics, such as Poly ADP ribose polymerase (PARP) inhibitors; cell cycle proliferation to assess the aggressiveness of prostate cancer; stability of tumor cells to a variety of cancer therapies to indicate whether a patient is likely to respond to the therapies.

The method of the present invention can also be used for determining the activity of small or large molecules on gene expression. In such cases, one or more small or large molecules are typically incubated with a cell sample prior to permeabilization and immersion into a mixture containing oligonucleotide probes. The effect of a molecule on gene expression can then be correlated with potentially therapeutic activity relative to a disease state.

The speed of imaging used in the present methods also permits high-throughput screening of small and/or large molecules as related to their effect on gene expression. Typically, at least 50 small (MW less than 1000 g/m) and/or large molecules (MW greater than 1000 g/m) can be screened in a 24 hour period using the same SAO system for imaging. In certain cases, 100, 150, 200, 250, 300, 350, 400, 450 or 500 small and/or large molecules can be screened.

The method of the present invention can further be used for genetic barcoding (e.g., DNA and RNA barcoding). In this way it can be used as a diagnostic method to rapidly recognize, identify and discover various species.

### Experimental Methods

The following materials, instrumentation and general methods are meant to illustrate aspects of methods of the present invention. They are not meant to limit the disclosed invention(s) in any way.

### Materials and Instrumentation

Oligomer probes are typically designed using an appropriate software package, such as Probe Designer, which is available at www.singlemoleculefish.com through Biosearch Technologies. The probes may be synthesized by any suitable method, including on an automated DNA/RNA synthesizer, e.g., Biosearch 8700.

Fluorophores are typically purchased from their respective suppliers. Nonlimiting examples of such fluorophores include: CAL FLUOR® and QUASAR® dyes, available from Biosearch Technologies; Cy3, Cy3.5, Cy5, available from Amersham; and, Oregon Green 488 and Alexa Fluor 488, available from Molecular Probes.

To attach a fluorescent label to the oligonucleotides, thereby producing singly-labeled probes, the oligonucleotides are pooled and coupled to a fluorophore in a single reaction, after which uncoupled oligonucleotides and remaining free fluorophores are removed by HPLC purification. See, US Pat. Publ. No. 2012/0129165 (Arjan Raj, et al.).

Glass slides may be purchased from any suitable supplier. A non-limiting example is Cat. No. 12-518-103 from Fisher.

Imaging of individual mRNA molecules using multiple singly labeled probes is typically accomplished using a system that performs synthetic aperture optics (SAO) on a target probe-mRNA hybrid. See, for example, International Publication Number WO 2011/116175. The performance specification for one system is as follows: Resolution—0.30 m; Imaging FOV—0.83 mm×0.7 mm; Working Distance—7.0 mm; Depth-of-Field—1.36 m; Sample Thickness—≤2 m; No. of z-sections—1-3; Target Medium—25 mm×75 mm substrate (e.g., microscope slide). “Resolution” is defined as the full-width-half-maximum (FWHM) of the point-spread-function (PSF) for 532 nm excitation and 600 nm emission wavelengths. Resolution is enhanced, for example, by using four beam, six beam or 10 beam delivery resolution. “Imaging FOC” is based on sCMOS camera (16.6 mm×14 mm sensor size) with 20× Objective magnification.

In terms of configuration of the SAO system, the following sub-systems and major components are typically used: Light Source—405 nm diode laser (100 mW), 532 nm laser (1 W, MPB/2RU-VFL-P-1000-532-R), 642 nm laser (1 W, MPB/2RU-VGL-P-1000-642-R); Illumination—Beam expander/combiner (LSG), Optical switch (Leoni/eol 1×4 PM) or free beam architecture, Pattern Generator (LSG); Imaging—OBJ-20×/0.45NA (Nikon MRH08230), Camera-sCMOS (Andor/DG152X-COE-FI), Filter wheel (10 slots, Sutter/Lamda 10-B), Filters (Samrock), PI-FOC (PI/P-725.4CD); Sample/storage—Z stage (motorized, PI/P-736.ZR2S), XY stage (motorized, PI/M26821LOJ), sample mount for slide or 35 mm dish (PI/P-545.SH3); Instrument control—Control board (LSG), Control software (LSG); Data analysis/UI—Analysis software (LSG); Main computer—Desktop computer (Dell/XPS8300); Table—Vibration isolation table (Newport/VIS3660-RG4-325A).

### Experimental Methods

The following is a non-limiting example of a method to prepare cell samples. See, Singer Lab Protocol, published online at www.singerlab.org/protocols.

Solution Preparation. Coverslips in 0.5% gelatin: A box of coverslips is sterilized by boiling them in 0.1N HCl for 20 min. The coverslips are rinsed and washed in doubly distilled water (“DDW”) several times. Gelatin (1.0 g) is weighed and added to 200 ml DDW. The resulting mixture is stirred and warmed to complete dissolution. Sterilized coverslips are transferred to the gelatin solution and autoclaved for 20 min. 10×PBS stock: To 500 ml of OX PBS is added 250 L DEPC. The mixture is stirred to dissolve and then autoclaved. 1 M MgCl2 stock. MgCl2 (20.3 g) is weighed and added to DDW. Washing solution (PBSM): To 100 ml 10×PBS stock is added 5 ml 1 M MgCl2 stock. The resulting mixture is diluted to 1 L with DDW. Extractant (PBST): To 100 ml 10×PBS stock is added 5 ml Triton X-100. The resulting mixture is diluted to 1 L with DDW and stir gently to complete dissolution. Fixative (4% PFA): To a 10 ml vial of 20% paraformaldehyde stock is added 5 ml 10×PBS stock. The resulting mixture is diluted to 50 ml with DDW.

Cell and Sample Preparation. Cells are grown under standard conditions and seeded onto gelatinized cover slips in a petri dish. Any treatment steps, such as starvation and stimulation, are performed. The cells are washed briefly with ice-cold PBSM. The cells are extracted in PBST for 60 seconds at room temperature. The cells are washed briefly with ice-cold PBSM twice. The cells are fixed with PFA fixative solution for 20 min. at room temperature. The cells are washed briefly with ice-cold PBSM twice. Fixed cover slips may be stored at 4° C. in PBSM until use.

The following is a non-limiting example of a method to hybridize oligonucleotide probes to target mRNA. See, Singer Lab Protocol, published online at www.singerlab.org/protocols. Also see, Femino A M, Fay F S, Fogarty K, and Singer R H. Visualization of single RNA transcripts in situ. 1998. Science. 280:585-90, and Levsky J M, Shenoy S M, Pezo R C and Singer R H. 2002. Single-cell gene expression profiling. Science. 297:836-40.

Solution Preparation. Washing solution (PBSM): To 100 ml 10×PBS stock is added 5 ml 1 M MgCl2 stock. The resulting mixture is diluted to 1 L with DDW. Pre/post-hybridization wash (50% formamide/2×SSC): To 250 ml formamide is added 50 ml 20×SSC stock. The resulting mixture is diluted to 500 ml with DDW. Probe competitor solution (ssDNA/tRNA): To 50 μl of 10 mg/ml sheared salmon sperm DNA is added 50 μl 10 mg/ml E. coli tRNA.

Hybridization buffer: To 60 μl DDW is added 20 μl BSA and 20 μl 20×SSC stock. Low-salt wash solution (2×SSC): To 50 ml 20×SSC stock is added 450 ml DDW. Nuclear stain solution (DAPI): To 100 ml 10×PBS stock is added 50 μl 10 mg/ml DAPI stock (prepared from solid by adding 10 mg to 1.0 ml DDW). The resulting mixture is diluted to 1 L with DDW and shaken to dissolve the DAPI. Mounting medium: Prepare ingredients of a suitable kit, such as the Prolong kit (Molecular Probes) or use an equivalent method.

Hybridization Steps. Hybridization is tested before color-coding and multiple transcript detection. Two bright dyes are used to show transcription sites. Each gene is subsequently assigned an arbitrary color code using combinations of dyes and tested singly. Fixed coverslips are placed vertically in a coplin jar using forceps. The fixed cells are rehydrated and washed in PBSM for ten min. at room temperature. Cells are equilibrated in pre-hybridization solution for 10 min. Aliquots of oligonucleotide probe mixtures are added to tubes for each different combination of targets to be assayed. Competitor solution is added to the probe mixture(s) in 100-fold excess. The mixture is vacuum dried. The dry pellet is re-suspended in 10 μl formamide and the tubes are placed on a heating block at 85° C. for 5-10 min and then immediately placed on ice. 10 μl of hybridization buffer is added to each tube, providing a reaction volume of 20 μl. A glass plate is wrapped with parafilm, allowing working space for the reactions. Each 20 μl reaction volume is dotted on the plate, far enough apart to allow cover slips to be place over each volume without overlap. The cover slips are removed from the pre-hybridization solution, and excess liquid is blotted off. Each cover slip is placed cell side down on the hybridization mix dotted onto the plate. Another layer of parafilm is wrapped over the plate and cover slips to seal the reactions. The plate is incubated at 37° C. for three hours, along with a sufficient amount of pre-hybridization solution to wash the cover slips twice after hybridization. The top layer of the parafilm is removed and the lower layer is lifted to allow removal of the cover slips. The cover slips are placed back into coplin jars with pre-warmed wash and incubated for 20 min. at 37° C. The wash is changed and repeated for 20 min. The solution is changed with 2×SSC and incubated at room temperature for ten min. The solution is changed with PBSM and incubated at room temperature for ten min. The nuclei are counterstained by changing the solution with prepared DAPI and incubating at room temperature for one min., then washing with PBSM. The PBSM is changed and kept at room temperature until mounting. Each coverslip is mounted cell-side down onto a glass slide, using freshly prepared antifade mounting medium The excess liquid is blotted off, and the slides are stored at −20° C.

Detection of oligonucleotide probe-target mRNA hybrids is performed with an SAO system as described above.

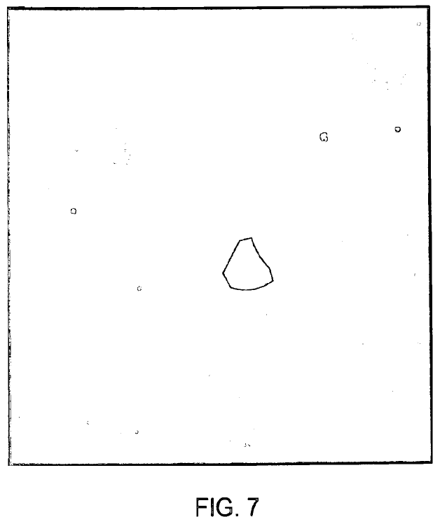

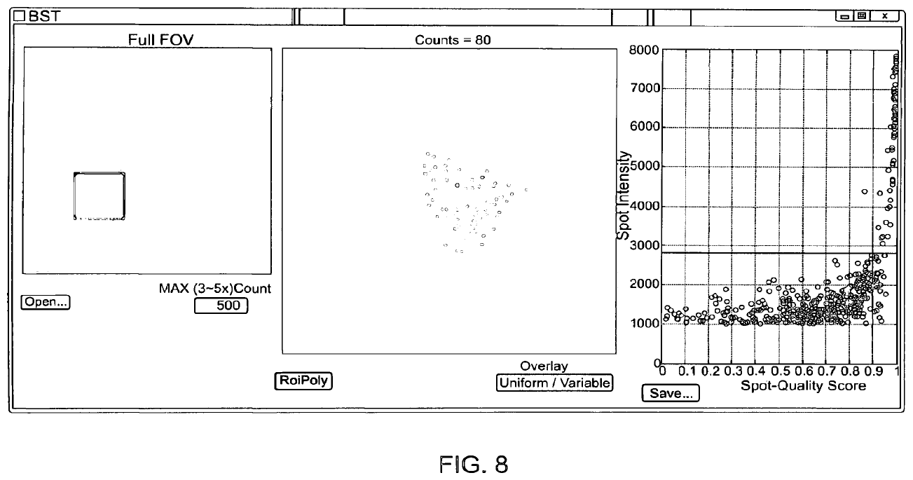

Quantification of TOP1 mRNA. Expression of TOP1 (topoisomerase (DNA) 1) was analyzed by FISH in A549 cells and imaged/quantified using an SAO system (20×). SAO imaging conditions were as follows: 500 mW main power (532 nm); 500 ms exposure per frame). A portion of the SAO image is shown in FIG. 7. The image includes approximately 100 cells, with the mRNAs appearing as bright/white/green dots in the image. A sampling of 20 cells within the image provided the following mRNA counts: 56; 59; 58; 54; 69; 60; 63; 54; 74; 65; 95; 52; 60; 85; 66; 67; 46; 36; 65; 53. FIG. 8 shows the selection of a region of interest of an SAO image of TOP1 mRNAs, including a selection process graph based on spot intensity and quality.

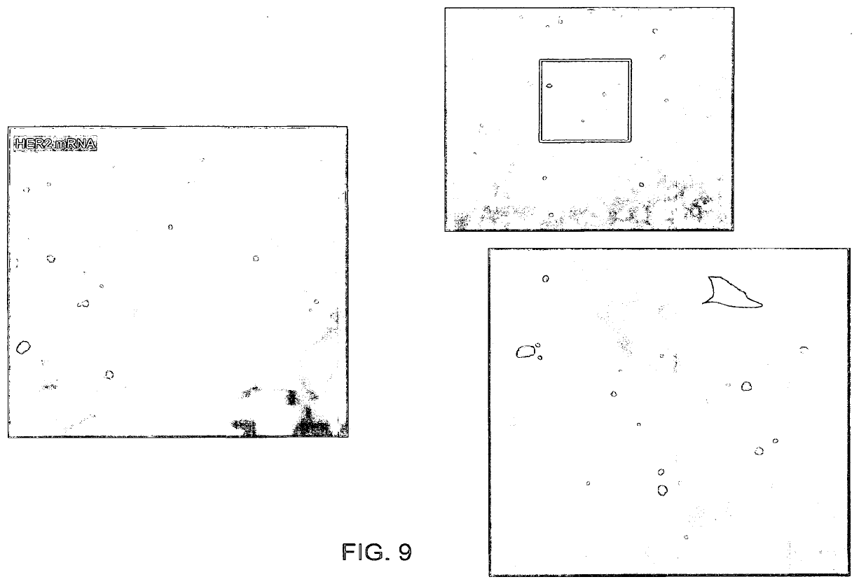

Quantification of HER2 mRNA. Expression of HER2 was analyzed by FISH in MCF7 cells (human breast adenocarcinoma cell line) and imaged/quantified using an SAO system (20×). SAO imaging conditions were as follows: 500 mW main power (532 nm); 500 ms exposure per frame). Results are shown in FIG. 9. The top right image includes over 100 cells, with the mRNAs appearing as bright/white dots in the image. The other images are a section showing approximately 20 cells. A sampling of 20 cells provided the following counts: 62, 61, 71, 97, 74, 66, 69, 48, 58, 87, 37, 92, 103, 80, 90, 21, 37, 109, 57, 122 (avg. 72).

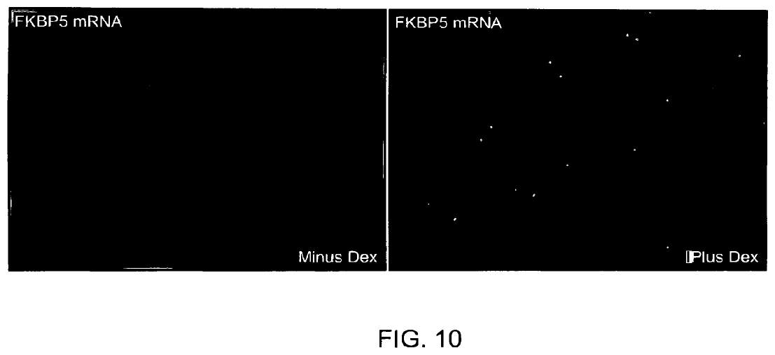

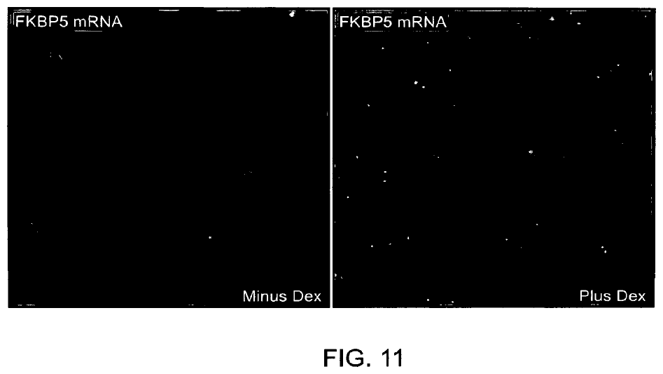

Quantification of FKBP5 mRNA. Expression of FKBP5 was analyzed by FISH in A549 cells (human lung adenocarcinoma cell line). FIG. 10 shows two images from a standard fluorescent microscope (60×/1.41 NA0.1 oil). The image labeled “Minus Dex” shows cells prior to upregulation by the addition of 24 nM dexamethasone (approximately 13 cells); the image “Plus Dex” shows cells after addition of 24 nM dexamethasone for 8 hours (approximately 14 cells). The larger, roughly oval structures are cell nuclei, with individual detected molecules shown as bright/white dots in and around the nuclei. FIG. 11 shows two images ( 1/10 of the full images obtained) using a system comprising an SAO imaging device (20×). The image labeled “Minus Dex” shows cells prior to upregulation by the addition of 24 nM dexamethasone (over 50 cells); the image “Plus Dex” shows cells after addition of 24 nM dexamethasone for 8 hours (over 50 cells). The larger, roughly oval structures are cell nuclei, with individual detected molecules shown as bright/white dots in and around the nuclei.

What is claimed is:

1. A method of imaging single molecules, the method comprising:
obtaining a sample that includes a plurality of complexes, a respective complex of the plurality of complexes including a target molecule bound to a probe, the probe including one or more fluorescent groups that interact with light at one or more wavelengths;
exposing an area of the sample to one or more wavelengths of light that interact with the one or more fluorescent groups, wherein an interference pattern is generated on the area of the sample in a region of overlap between two or more coherent laser beams; and
detecting a result from the interacting of the one or more wavelengths of light that interact with the one or more fluorescent groups using 20× objective magnification to provide an image of one or more single molecules without variation of any detection settings so that the image possesses a resolution better than 450 nm over the imaged area of at least 1×10⁵ μm².

2. The method of claim 1, wherein:
the interference pattern is generated on the area of the sample in the region of overlap of at least four paired coherent laser beams.

3. The method of claim 1, wherein:
the two or more coherent laser beams are focused toward the area of the sample using the 20× objective magnification.

4. The method of claim 1, further comprising:
quantifying the single molecules from the image having the imaged area of at least 1×10⁵ μm².

5. The method of claim 1, wherein:
the targeted molecule is selected from a group consisting of mRNAs, lnc RNAs, snRNAs, a chromosome, a DNA strand comprising BrdU, a DNA strand comprising EdU, a protein, and a small molecule.

6. The method of claim 1, wherein:
the one or more fluorescent groups include a fluorescent compound selected from a group consisting of fluorescent organic dyes, quantum dots, intercalator fluorescent dyes and expressible fluorescent proteins.

7. The method of claim 1, wherein:
the density of the one or more fluorescent groups within the field of view is less than 1000 molecules per m².

8. The method of claim 1, wherein:
the imaged area is at least 1×10⁶ μm².

9. The method of claim 1, wherein:
the sample contains a plurality of cells including a plurality of target molecules.

10. The method of claim 9, wherein:
exposing live cells in the sample to a plurality of probes to form the plurality of complexes.

11. The method of claim 10, wherein:
a respective probe of the plurality of probes includes a first portion that specifically binds to a target molecule and a second portion that is modifiable to include the one or more fluorescent groups that interact with light at one or more wavelengths.

12. The method of claim 11, further comprising:
after the respective probe specifically binds to a target molecule, modifying the second portion of the respective probe to include the one or more fluorescent groups that interact with light at one or more wavelengths.

13. The method of claim 11, wherein:
the second portion of the probe is modified using a type of chemical reaction selected from a group of chemical reactions consisting of: Click chemistry; a Diels-Alder reaction; Staudinger ligation; hydrazine ligation; oxime ligation; native chemical ligation; tetrazine ligation; maleimide-thiol ligation; active ester-amine ligation; carbodiimide phosphate conjugation; and, carboxy conjugation.

14. The method of claim 9, wherein:
the plurality of target molecules includes an mRNA molecule; and
probes in the sample include a plurality of oligonucleotides that are capable of hybridizing to the mRNA molecules, each oligonucleotide including a single fluorescent label, providing a set of singly-labeled oligonucleotides to afford a set of oligonucleotide-mRNA hybridized products.

15. The method of claim 9, wherein:
the plurality of target molecules includes an lnc RNA molecule; and
probes in the sample include a plurality of oligonucleotides that are capable of hybridizing to the lnc RNA molecules, each oligonucleotide including a single fluorescent label, providing a set of singly-labeled oligonucleotides to afford a set of oligonucleotide-lnc RNA hybridized products.

16. The method of claim 9, wherein:
the plurality of target molecules includes an snRNA molecule; and
probes in the sample include a plurality of oligonucleotides that are capable of hybridizing to the snRNA molecules, each oligonucleotide including a single fluorescent label, providing a set of singly-labeled oligonucleotides to afford a set of oligonucleotide-snRNA hybridized products.

17. The method of claim 9, wherein:
the plurality of target molecules includes a chromosome or a portion of a chromosome; and
probes in the sample include a plurality of oligonucleotides that are capable of hybridizing to the chromosome or the portion of the chromosome, each oligonucleotide including a single fluorescent label, providing a set of singly-labeled oligonucleotides to afford a set of oligonucleotide-chromosome hybridized products.

18. The method of claim 9, wherein:
the target molecule is an mRNA selected from a group consisting of: CCNB1 mRNA, CENPE mRNA, AURKB mRNA, PLK1 mRNA, PLK4 mRNA, TAGLN mRNA, ACTG2 mRNA, TPM1 mRNA, MYH111 mRNA, DES mRNA, EF1AX mRNA, AR mRNA, HSPD1 mRNA, HSPCA mRNA, K-ALPHA1 mRNA, MLL5 mRNA, UGT2B15 mRNA, WNT5B5 mRNA, ANXA11 mRNA, FOS mRNA, SFRP1 mRNA, FN1 mRNA, ITGB8 mRNA, THBS2 mRNA, HNT mRNA, CDH10 mRNA, BMP4 mRNA, ANKH mRNA, SEP4 mRNA, SEP7 mRNA, PTN mRNA, VEGF mRNA, SRY mRNA, EGR3 mRNA, FoxP1 mRNA, FoxM1 mRNA, TGCT1 mRNA, ITPKB mRNA, RGS4 mRNA, and BACE1 mRNA.

19. The method of claim 9, wherein:
the target molecule is BrdU incorporated into a replicating DNA strand of a cell;
the probes in the sample include an anti-BrdU antibody comprising one or more fluorescent groups; and
the method includes:
providing an amount of BrdU to the plurality of live cells;
incubating the provided BrdU with the plurality of live cells for a time period that allows for a significant amount of the BrdU to be incorporated into proliferating cells;
providing an amount of the anti-BrdU antibody to the plurality of cells incorporating the BrdU; and
incubating the provided antibody with the plurality of live cells incorporating the BrdU for a time period that allows for binding of a significant amount of the anti-BrdU antibody to the BrdU incorporated into the plurality of cells.

20. The method of claim 9, wherein:
the target molecule is EdU incorporated into a replicating DNA strand of a cell;
the probes in the sample include an amount of a fluorescently labeled, azide-based Click reagent; and
the method includes:
providing an amount of EdU to the plurality of live cells;
incubating the provided EdU with the plurality of live cells for a time period that allows for a significant amount of the EdU to be incorporated into proliferating cells; and
providing an amount of the fluorescently labeled, azide-based Click reagent under conditions that allow reaction between the incorporated EdU and the Click reagent.
